(19) United States
(12) Patent Application Publication
(10) Pub. No.: US 2024/0350159 A1
(43) Pub. Date: Oct. 24, 2024

(54) SURGICAL INSTRUMENT AND CONTROL MECHANISM THEREFOR

(71) Applicant: KARL STORZ SE & CO. KG, Tuttlingen (DE)

(72) Inventors: Jochen STEFAN, Wald (DE); Sven Axel GRÜNER, Trossingen (DE)

(73) Assignee: KARL STORZ SE & CO. KG, Tuttlingen (DE)

(21) Appl. No.: 18/291,415

(22) PCT Filed: Jul. 25, 2022
(86) PCT No.: PCT/EP2022/070836

§ 371 Date: Jan. 23, 2024

(30) Foreign Application Priority Data
Jul. 28, 2021 (DE) 10 2021 119 533.0

(51) Int. Cl. A61B 17/29; A61B 17/00; A61B 34/00; A61B 34/30

(52) U.S. Cl. CPC A61B 17/29; A61B 34/30; A61B 34/70; A61B 2017/00398; A61B 2017/00477; A61B 2017/00858; A61B 2017/00862; A61B 2017/2903; A61B 2017/292; A61B 2017/2929

(57) ABSTRACT

A control mechanism for a surgical instrument including a shaft, and an angling mechanism for actuating a tool. The control mechanism has a shaft element which forms an extension of the shaft when the control mechanism and the surgical instrument are assembled. The control mechanism includes a control ring, operatively coupled to the angling mechanism and has a central-axial through-opening for a bearing device which can be rotated about the longitudinal axis together with the shaft element. Three motorised drives are operatively coupled to the control ring to transmit the adjustment angle of the three drives to the control ring, wherein the control ring can be aligned to one another and to the longitudinal axis, via the adjustment angle of two of the three drives, and can be rotated about the longitudinal axis via the adjustment angle of the third of the three drives.

### CROSS-REFERENCE TO RELATED APPLICATIONS

This application is the U.S. national stage of PCT/EP2022/070836 filed on Jul. 25, 2022, which claims priority of German Patent Application No. 10 2021 119 533.0 filed on Jul. 28, 2021, the contents of which are incorporated herein.

### TECHNICAL FIELD

The disclosure relates to a steering gear of a surgical instrument for deflecting a tool tip at an angle by means of a spatially orientable wobble steering ring, and a surgical instrument which comprises such a steering gear.

### BACKGROUND

From the prior art, surgical instruments are known which can be guided manually or by a robot and which have tools whose tool tip can be pivoted by means of a plurality of pivot members engaging in one another. These pivot members are connected to a multiplicity of steering wires or steering cables in order to achieve delicate control of the tool tip. A more uniform force distribution in all deflection directions can be obtained by way of a plurality of thin steering wires in comparison with a few thicker steering wires

It is known from U.S. Pat. No. 5,454,827 B2 to couple such steering wires to a spatially adjustable wobble plate arranged proximally in a manipulation unit, which wobble plate is connected via a rod to a manually manipulable control lever, such that a movement of the spatially adjustable wobble plate causes a corresponding relative movement of the distal-side pivot members and thus a pivoting of the tool tip.

It is also known, in a surgical instrument with a compact steering gear, to transmit the adjustment angles of two drives directly to the wobble plate in order to orient the latter for controlling the tool tip. To do this, steering wires are fastened to the wobble plate so that the tip of the tool can be steplessly and smoothly controlled by alignment of the wobble plate. For this purpose, the known steering gear has two drive bevel gears offset by 180° from one another, which are arranged on a common axis of rotation that runs perpendicular to an instrument longitudinal axis, each with an associated motor. The wobble plate is arranged between the drive bevel gears and is mounted in a steering ring which is connected for conjoint rotation to a third bevel gear which engages with the two drive bevel gears and is rotatable about an axis of rotation perpendicular to the instrument longitudinal axis and perpendicular to the common axis of rotation of the drive bevel gears. The gear chain is supplemented by a fourth bevel gear, which is arranged on the axis of rotation of the third bevel gear, offset by 180° from the third bevel gear, and is in engagement with the two drive bevel gears, wherein the steering ring is mounted freely rotatably in the fourth bevel gear. The gear chain, closed in this way, ensures that all the bevel gears engage with one another and permits uniform force distribution.

The design of the drive for the steering wires with the spatially adjustable wobble plate, on which the steering wires are mounted, is advantageous in that this enables a spatially compact structure and only requires the movement of one component in order to be able to address all the steering wires.

U.S. Pat. No. 7,699,855 B2 discloses a surgical instrument which has an interface so as to be able to connect the instrument to a robot arm. All the drives that control the instrument are arranged in the robot arm. The rotation angles are transferred from drives to the instrument via clutch disks in a common separation plane.

WO 2014/004242 also describes such an interface, wherein the drives are in-stalled in the robot arm.

The above design is associated with a complex structure and with an indirect control. The drives are not directly arranged in the surgical instrument, which means that the wobble plate is not controlled linearly.

U.S. Pat. No. 10,105,128 B2 also discloses a control of such a tool tip; in that case, this is implemented by way of a mechanism comprising toothed lock washer segments and joint rods for transmitting the movement of the drives to the wobble plate.

### SUMMARY

Proceeding from this prior art, it is an object of the present disclosure to provide alternative guidance of the steering ring.

This object is achieved by a steering gear having the features of claim 1.

The further object of making available a surgical instrument with alternative guidance of the steering ring is achieved by the surgical instrument having the features of independent claim 11.

Developments and preferred embodiments of the steering gear and the surgical instrument are defined in the dependent claims.

The steering gear according to the disclosure is designed for the handling of a surgical instrument which comprises a shaft, at the distal end of which a deflection mechanism for manipulating a tool is present. The steering gear according to the disclosure can be arranged at the proximal end of the shaft, which has a longitudinal axis B. According to a first embodiment, the steering gear according to the disclosure comprises a shaft element which likewise has a longitudinal axis and which continues the shaft in the case of an assembled arrangement, in which the steering gear and the surgical instrument are assembled on one another: accordingly, the two longitudinal axes also continue.

According to the disclosure, the steering gear in this case comprises a wobble steering ring which can be operatively coupled to the distal deflection mechanism for controlling the deflection of same. The wobble steering ring has a central axial through-opening for a bearing device which is secured in the axial longitudinal direction on the shaft element of the steering gear and rotatable about the longitudinal axis B with the shaft element.

According to the disclosure, the steering gear comprises three motorized drives and provides an operative coupling of the three drives to the wobble steering ring for transferring the adjustment angles of the three drives to the wobble steering ring. In this case, the wobble steering ring can be spatially alignable in relation to two pivot axes running perpendicular to one another and to the longitudinal axis by way of the adjustment angles of two of the three drives and can be rotated about the longitudinal axis by way of the adjustment angle of the third of three drives.

In this case, the longitudinal axis of the rod-type shaft is identical to the longitudinal axis of the shaft of the surgical instrument into which the steering guide according to the disclosure can be inserted. The shaft element which can be arranged at the proximal end of the shaft of the surgical instrument and which can continue this shaft enables an axial installation space that is as short as possible and hence enables a space-saving, compact steering gear.

Advantageously, the wobble steering ring can be controlled directly by means of the drive motors. No further diversion mechanisms or gear transmissions are necessary, and therefore the shortest possible transmission chain is permitted. Such a direct force transmission which exhibits a linear transmission behavior allows simple software control, with the result that precise and reliable control of the component to be controlled is achieved.

According to a further embodiment of the steering gear according to the disclosure, the bearing device of the wobble steering ring rotatable with the shaft element about the longitudinal axis is a gimbal-type bearing device which comprises a universal joint plate which is arranged in the central axial through-opening and comprises at least four radial drilled through holes offset by 90° from one another in each case, wherein a bearing pin is arranged in each radial drilled through hole. Two first bearing pins which are arranged coaxially with respect to one another, which is to say arranged offset by 180° from one another, in the drilled through holes and which form a first bearing pin pair connect the wobble steering ring to the universal joint plate. The two further second bearing pins which are arranged coaxially with respect to one another, which is to say arranged offset by 180° from one another, in the drilled through holes and which form a second bearing pin pair connect the universal joint plate to the shaft element, wherein the first bearing pin pair is present 90° offset from the second bearing pin pair. Consequently, the universal joint plate is pivotably arranged on the shaft element by means of the bearing pin pairs and mounts the wobble steering ring such that the latter can be tilted about the pivot axes and rotated with the shaft element about the longitudinal axis B. Hence, the wobble steering ring is laterally fixed in space on the shaft element, which is rotatably mounted at both ends, but can be tilted or rotated in all three spatial directions, whereby the tool tip can be controlled in a targeted manner.

In yet a further embodiment of the steering gear according to the disclosure, the wobble steering ring has a spherical disk-shaped outer lateral surface as effecting surface. Further, the steering gear has two longitudinally axially displaceable gear shafts, each with a contact roller portion, wherein the gear shafts each have an axis of rotation which is parallel to the longitudinal axis of the shaft element and spans a center angle of 90° with the longitudinal axis. In this case, the respective contact roller portion contacts the effecting surface of the wobble steering ring at a contact point tangentially, wherein the two pivot axes are each defined by a straight line which runs through the center of the spherical disk-shaped outer lateral surface and through the respective contact point of the respective contact roller portion with the effecting surface. The contact roller portion acts as a force transmitter, starting from the movement of the gear shaft to the effecting portion of the wobble steering ring.

In this context, “spherical disk-shaped” refers to a torus-like, partial sphere-shaped or else toroidal outer lateral surface defined by a surface of revolution formed by rotating an outwardly arched circular arc about the longitudinal axis, wherein the circle center of the all-round circular arc is located on the longitudinal axis and hence each contact point is ideally equidistant from the circle center.

Here, “effecting portion” is the region of the steering ring that can enter into a force-transmitting operative connection with the contact roller portion, which is to say is directly in contact with the force transmitter, for example by means of a frictional operative connection using friction elements, or that is engaged therewith, for example by means of a toothing or other suitable force-transmitting operative connections. Thus, instead of by way of friction, the force transmission can alternatively be implemented by way of a mutual, meshing toothing as an operative connection.

Here, “contact roller portion” refers to any component that directly transmits the movement initiated by motors, whether rotational or linear, which is to say is able to pass said movement on to the effecting portion of the steering ring in the sense of a force transmission. Alternatively, instead of by way of friction, the force transmission can be implemented by way of a meshing toothing or any other suitable operative connection.

In an alternative embodiment of the steering gear according to the disclosure, the steering gear may also comprise four gear shafts, respectively arranged with a 90° offset around the wobble steering ring and all rotationally driven in the same sense by the third motor. In this case, all gear shafts would have to rotate in the same sense for the purpose of rotating the wobble steering ring, and gear shafts arranged in pairs or opposite one another in each case would have to rotate in the opposite sense for the purpose of a tilt. This can achieve a further stable movement of the gear, and hence of the control of the wobble steering ring, since twice as many contact roller portions with greater traction can apply greater contact pressure and hence more force on the wobble steering ring.

A further embodiment of the steering gear according to the disclosure provides for each gear shaft to have a drive portion which comprises a toothed rod portion with teeth all around the circumference, which defines an axial displacement path of each gear shaft and meshes with a pinion (drive pinion) seated on a drive shaft of the respective motor, wherein the drive shafts define drive axes which run perpendicular to the longitudinal axis and the axes of rotation of the gear shafts. A movement of a gear shaft initiated by controlling one of the first two motors causes the drive pinion to mesh in the spur toothing of the drive portion, whereby the gear shaft is displaced linearly within the steering gear, depending on the direction in which the drive shaft rotates the pinion, for example pushed back and forth in the distal direction. Accordingly, the contact roller portion is also pushed forwards or backwards in relation to a distal end of the steering gear. As a result of the friction present between the contact roller portion and the lateral surface of the wobble steering ring, which is to say the effecting portion thereof, the wobble steering ring is tilted at this point, to be precise about its respective tilt axis, by targeted force transmission.

Here, “drive portion” refers to any component that directly receives the movement initiated by motors, whether rotational or linear, and converts said movement into a movement of the gear shafts.

In a further embodiment of the steering gear according to the disclosure, the two gear shafts are driven to rotate in the same sense about the respective axis of rotation by the third motor. In this case, each gear shaft preferably comprises a gear wheel at a free end of the drive portion, the gear wheel meshing with a common drive gear wheel driven by the third motor by way of a drive shaft parallel to the longitudinal axis and the axes of rotation. The gear shafts are therefore driven together by the third motor, whereby the wobble steering ring can rotate. As a result of contact roller portions running in the same sense, the gear shafts allow the rotation of the wobble steering ring and, if the contact roller portions are in the form of rollers, form a type of roller mill, whose drum corresponds to the wobble steering ring.

In yet a further embodiment of the steering gear according to the disclosure, a force transmission between the contact roller portions of the gear shafts and the effecting portion of the wobble steering ring is provided by friction. One or both of the friction partners, consisting of the effecting portion of the wobble steering ring and the respective contact roller portion, comprise a cladding made of a friction-increasing material. Alternatively, they consist in full of the friction-increasing material. Further, the wobble steering ring may comprise such material or only the contact roller portions or both components. Friction enables a structurally simple force transmission, by way of an appropriately selected material which applies a certain frictional force. According to a simple variant, these contact roller portions can be claddings, for example pulled-on tubing portions made of an elastomer such as rubber for example.

According to yet a further embodiment of the steering gear according to the disclosure, the steering gear comprises a bearing housing with a base plate arranged below the two gear shafts and parallel to the latter. In this case, the bearing housing has a first side plate fastened to the base plate at the end of the contact portions of the gear shafts and comprising two drilled passage holes for mounting the gear shafts. Moreover, the bearing housing comprises a second side plate with two drilled passage holes, through which the drive portion located between the toothing portions and the gear wheels at the end extends and which is fastened to the base plate at the end of the latter distant from the first side plate. The length of the portion of the gear shaft between the toothing portion and the gear wheel at the end at least corresponds to the length of the displacement path of the gear shaft, with the result that neither the gear wheel nor the toothing portion impact on the second side plate during the axial movement of the gear shaft. The bearing housing enables secure bearing of the movable components of the steering gear. The shaft element is also rotatably mounted in these two components by means of two ball bearings.

According to yet a further embodiment of the steering gear according to the disclosure, the wobble steering ring comprises a first end face which has a first funnel-shaped cutout. Drilled passage holes for leading through steering wires are present in a base of the first funnel-shaped cutout. The steering wires are preferably clamp-mounted on the wobble steering ring by means of a clamping connection so that, in the case of damage, the steering wires can easily be replaced. For example, this clamped connection can be provided by a clamping disk which can be arranged in the second funnel-shaped cutout. The steering wires can also be adhesively bonded, welded or soldered to the wobble steering ring, in order to obtain a stable attachment of the steering wires. As a result of the steering wires being able to be connected directly to the wobble steering ring, there is no need for any further additional components, for example a separate arrangement of the wobble plate in a steering ring, as was previously the case in the prior art.

Further, the central axial through-opening of the wobble steering ring extends through the funnel base of the first funnel-shaped cutout. Further, the wobble steering ring comprises a second end face which has a second funnel-shaped cutout. The second funnel-shaped cutout has a smaller diameter than the first funnel-shaped cutout. In this case, the central axial through-opening extends further through the funnel base of the second funnel-shaped cutout such that a continuous linear through-opening through the wobble steering ring is present around the geometric center of the latter, the gimbal-type bearing device for mounting the wobble steering ring on the shaft element being able to be inserted into said through-opening. As a result of the funnel-shaped cutouts, the wobble steering ring can be tilted flexibly in space without interfering with the course of the steering wires: these can readily follow the movement of the wobble steering ring.

A first embodiment of a surgical instrument according to the disclosure, which comprises a shaft, a manipulation unit arranged at the proximal end of the shaft, and a tool arranged at the distal end of the shaft with a tool tip able to be deflected by means of a distal deflection mechanism, makes reference to the fact that the tool tip able to be deflected can be aligned in space by way of a steering gear according to the disclosure.

By virtue of the steering gear according to the disclosure, the surgical instrument can be constructed in structurally simple and space-saving fashion, with the result that a simple connection to a robotic arm can be enabled, in the case of which the movement of the drives can be transmitted directly to the tool tip. The consequence is a precisely controllable use of the surgical instrument.

According to a further embodiment of the surgical instrument according to the disclosure, the manipulation element is axially displaceably mounted in the shaft and is operatively connected to the manipulation unit on the proximal side. The distal deflection mechanism of the tool tip able to be deflected consists of pivot members which are arranged at the distal end of the shaft and are connected to the steering gear via the steering wires running in the longitudinal direction of the shaft. Compared to known constructions of the surgical instrument, this construction is advantageous not only in that it is possible to use a small number of steering wires, specifically just four steering wires, and in that the spatially adjustable plate serving as a drive for the steering wires can be manipulated exclusively manually, but also in that a plurality of steering wires can be chosen freely, thereby enabling sensitive and reproducible adjustment of the distal-side pivot members.

The surgical instrument according to the disclosure has the advantage that many thin steering wires can be used to control the pivotable tool tip and that, on account of the motorized drive for the spatially adjustable plate on which the steering wires are mounted proximally, this control is sensitive, precise and reproducible.

Further embodiments, and some of the advantages connected to these and further embodiments of the steering gear and surgical instrument, are rendered clear and better understandable by the following detailed description which makes reference to the attached figures. Objects or parts thereof which are substantially the same or similar may be provided with the same reference signs. The figures are merely a schematic illustration of an embodiment of the disclosure. The drawings, the description and the claims contain numerous features in combination. A person skilled in the art will advantageously also consider the features on an individual basis and combine them to form further advantageous combinations.

### BRIEF DESCRIPTION OF DRAWINGS

FIG. 1 shows a schematic perspective side view of a surgical instrument,

FIG. 2 shows a perspective view of a steering gear according to the invention with a wobble steering ring,

FIG. 3 shows a further perspective, partial cut view of a steering gear according to the invention with a wobble steering ring,

FIG. 4 shows a plan view of the steering gear according to the invention,

FIG. 5 shows a lateral sectional view along the longitudinal axis B through the steering gear according to the invention,

FIG. 6 shows a front view of the steering gear according to the invention,

FIG. 7 shows a front view of the wobble steering ring with the force transmitters of the gear shafts, and

FIG. 8 shows a sectional view of the wobble steering ring.

### DETAILED DESCRIPTION OF PREFERRED EMBODIMENTS

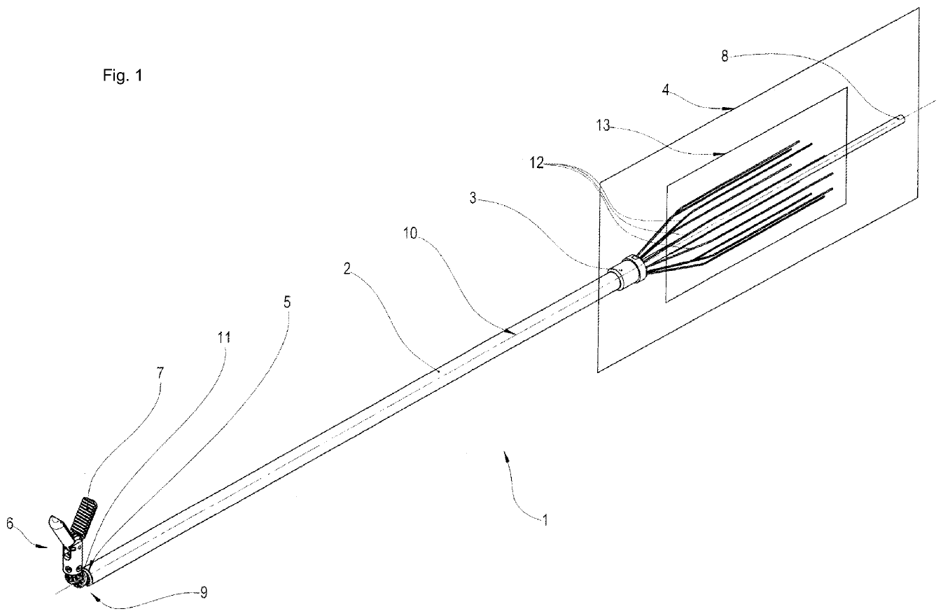

FIG. 1 shows schematically a surgical instrument 1 having a hollow shaft 2, a manipulation unit 4 (depicted only schematically) arranged at the proximal end 3 of the shaft 2, and a tool tip 6, arranged at the distal end 5 of the shaft 2 and comprising a tool 7 manipulable by way of a manipulation element 8 axially displaceably mounted in the shaft 2, said manipulation element, on the proximal side, being operatively connected to the manipulation unit 4. The manipulation unit 4 can be a manually manipulable handle, or else a component designed for robotic use, which is to say a component that is manipulable without manual assistance as well. For example, the tool 7 at the tool tip 6 can be a tool provided with jaws, as depicted in FIG. 1, or else alternatively an endoscope, an applicator, or the like (not shown in the figures). The tool tip 6 is pivotable relative to the longitudinal axis 10 of the shaft 2 by way of a joint mechanism 9, wherein the joint mechanism 9 consists of pivot members 11 which are arranged at the distal end of the shaft 5 and connected via steering wires 12 running in the longitudinal direction of the shaft 2 to a drive in the form of a steering gear 13 arranged at the proximal end 3 of the shaft 2, in such a way that a movement of the proximal-side drive 13 causes a corresponding relative movement of the distal-side pivot members 11 and hence a pivoting of the tool tip 6. Even though exclusive use is made of the term steering wires 12 hereinabove and below; from a functional point of view use can also be made of steering cables, which is why the used term steering wires 12 should also be read and understood synonymously as steering cables.

The manipulation element 8, which is mounted axially displaceably in the shaft 2 and serves to manipulate the tool tip 6 of the tool 7, is in the form of a push/pull rod in the embodiments depicted. The steering gear 13 for the steering wires 12 is a motorized steering gear 13 which comprises a spatially adjustable wobble steering ring 14 on which the steering wires 12 are mounted such that a displacement of the wobble steering ring 14, by preference brought about by way of the motorized steering gear 13, brings about a pivoting of the tool tip 6 by way of the steering wires 12.

In the surgical instrument 1 according to FIG. 1, the shaft 2 is connected to the motorized steering gear 13 by way of a shaft element 21 which is arranged coaxially with the longitudinal axis B of the shaft 2 and which is arranged at the proximal end 3 of the shaft 2 as a rod-type shaft 21 which is rotatable about the longitudinal axis B of the shaft 2. The manipulation element 8 for manipulating the instrument 7 is axially displaceably mounted within the shaft 2 and the adjoining shaft element 21.

The steering wires 12 emerging from the shaft 2 at the proximal end 3 of the shaft 2 are fed to and secured on a wobble steering ring 14 mounted on the shaft element 21. To this end, the wobble steering ring 14 has an axially parallel drilled through hole 30 for each steering wire 12, wherein the steering wires 12 are fastened within the drilled through holes 30.

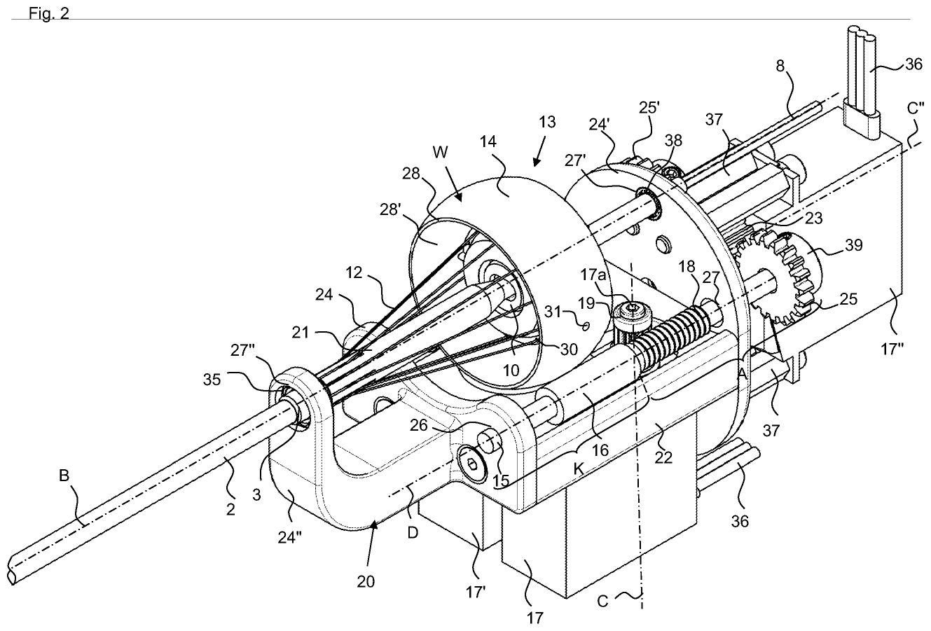

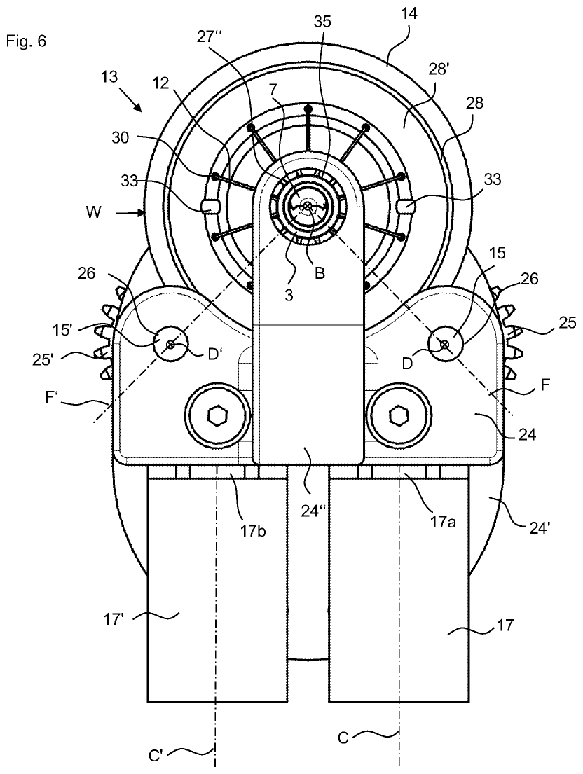

FIGS. 2 to 6 show the steering gear 13 of an embodiment according to the disclosure in detail, the core being the spatially adjustable wobble steering ring 14, to which the steering wires 12 are fastened such that a displacement of the wobble steering ring 14 brings about a pivoting of the tool tip 6 by way of the steering wires 12 mounted on the wobble steering ring 14. The steering wires 12 for pivoting the distal-side pivot members 11 or tool tip 6 can be controlled precisely, delicately in very small increments, and also in reproducible fashion by means of the structure by way of the wobble steering ring 14. Moreover, the number of steering wires 12 to be used for a motorized drive 13 can be chosen quite freely; in the depicted example, for example FIG. 6, the wobble steering ring 14 provides ten drilled through holes 30 for the steering wires 12.

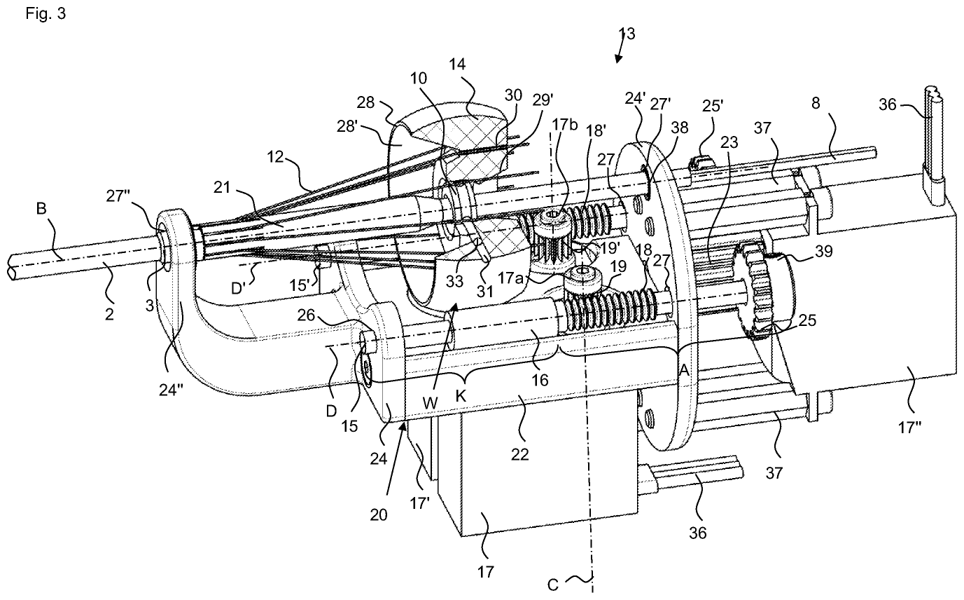

As can be seen in FIGS. 2 and 3, the steering gear 13 comprises three drives having motors 17, 17′, 17″ with drive axes C, C′, C″, wherein the drive axes C, C′ of the first motor 17 and second motor 17′ run parallel to one another and at right angles to the longitudinal axis B. The drive axis C″ of the third motor 17″ runs parallel to the longitudinal axis B. The longitudinal axis B is the common axis in the longitudinal direction of the surgical instrument 1 according to FIG. 1 and of the steering gear 13. Therein, the shaft 2 of the surgical instrument 1 is located on the longitudinal axis B, and also a shaft element 21 which adjoins the shaft 2 and on which the wobble steering ring 14 is arranged.

The first two motors 17, 17′ each have a drive shaft 17a, 17b, on each of which a respective pinion 19, 19′ is seated. The pinions 19, 19′ each engage with a respective gear shaft 15, 15′, wherein each gear shaft 15, 15′ comprises a contact roller portion K and a drive portion A. The gear shafts 15, 15′ each have an axis of rotation D, D′ each of which is parallel to the longitudinal axis B and spans a center angle of 90° with the longitudinal axis B, as depicted in FIG. 6.

The drive portion A of each gear shaft 15, 15′ comprises a toothed rod portion 18, 18′ with teeth all around the circumference, with which each pinion 19, 19′ meshes. The toothing is straight such that the pinions 19, 19′ also rotate when one or both motors 17, 17′ rotate their drive shafts 17a, 17b, wherein the gear shafts 15, 15′ are displaced linearly, in the distal or proximal direction depending on the direction of rotation of the drive shafts 17a. 17b, on account of the constant engagement in the respective toothed rod portions 18, 18′ of the gear shafts 15, 15′. The toothed rod portion 18, 18′ has a length corresponding to an axial displacement path of the gear shaft 15, 15′.

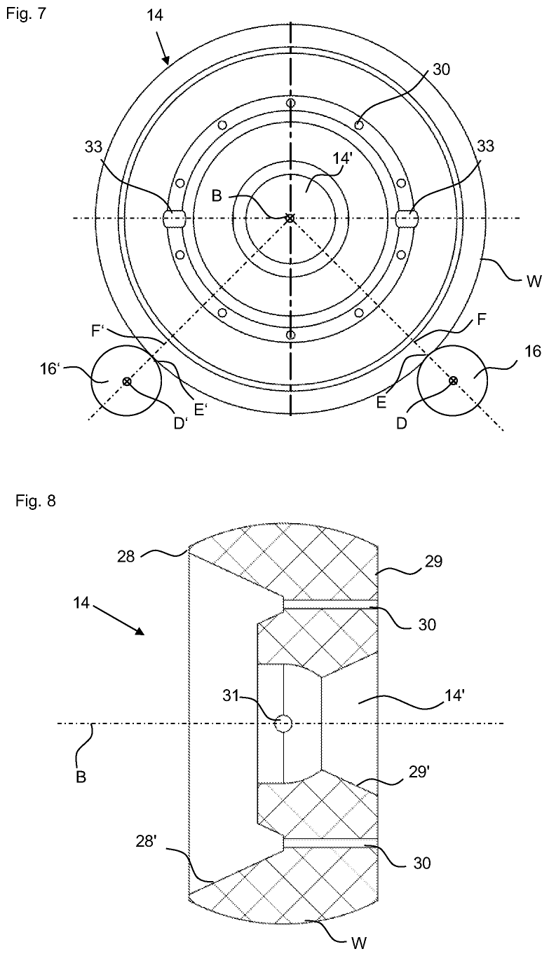

The respective contact roller portion K of the gear shafts 15, 15′ is seated in a distal region of the gear shafts 15, 15′ and tangentially contacts a lateral surface of the wobble steering ring 14 at a contact point E, E′ on an effecting surface W, as may be seen from FIG. 7 in particular. The effecting surface W corresponds to the entire lateral surface of the wobble steering ring 14. The geometric shape of the wobble steering ring 14 is a symmetrical spherical disk, the geometric center of which is located on the longitudinal axis B. Together with the axes of rotation D, D′ of the gear shafts 15, 15′, the center forms an equilateral triangle whose sides are in each case defined by a straight line running perpendicular to the longitudinal axis B and the respective axis of rotation D, D′ through the center of the spherical disk and the contact point E, E′ on the effecting surface W of the wobble steering ring 14 and which in each case defines a gimbal pivot axis F, F′, which include a right angle at the center. The wobble steering ring 14 can be tilted in all three spatial directions about these pivot axes F. F′.

To transmit the linear movement of the gear shafts 15, 15′ to the wobble steering ring 14, force transmitters 16, 16′ are provided in the contact roller portion K, said force transmitters, as all-round rubber lining, bringing about the force transmission by way of friction. A material with a high coefficient of friction increases the traction and hence the force transmittable to the effecting surface W, both for tilt and rotation.

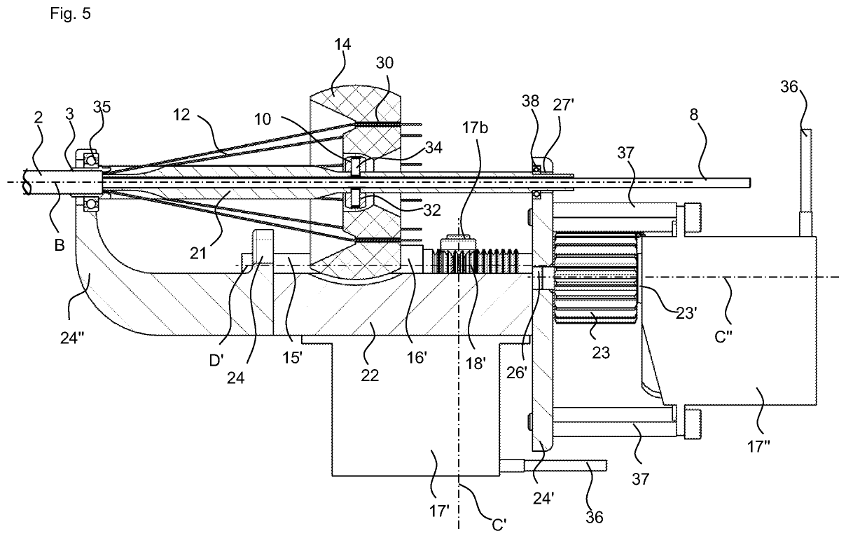

The two gear shafts 15, 15′ are driven to rotate in the same sense about the respective axis of rotation D, D′ by the third motor 17″. To this end, each gear shaft 15, 15′ comprises, at its free, proximal end of the drive portion A, a gear wheel 25, 25′ which meshes with a common drive gear wheel 23. The common drive gear wheel 23 is driven by the third motor 17″ by way of a drive shaft 23′ which is parallel to the longitudinal axis B and the axes of rotation D, D′ and which defines the third drive axis C″ (cf. FIG. 5).

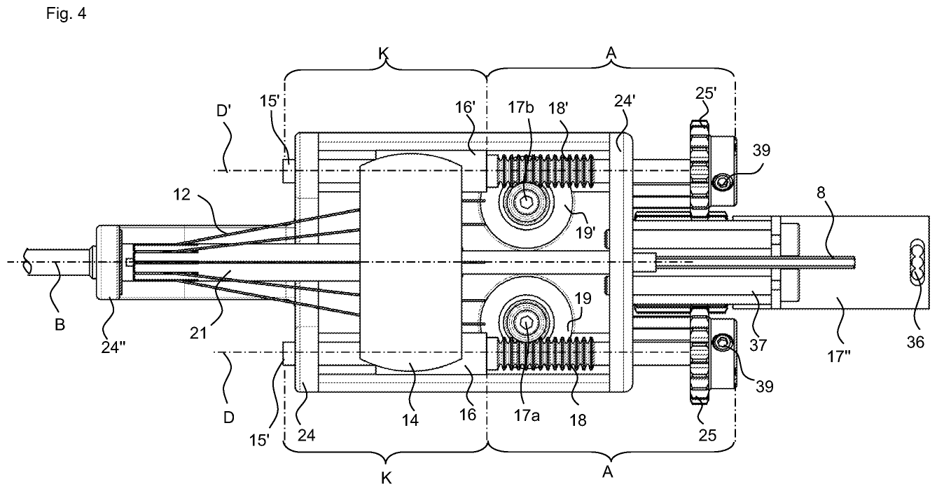

An axial length of the drive gear wheel 23 is adapted to the axial displacement path of each gear shaft 15, 15′ such that the gear wheels 25, 25′ continue to mesh with the common drive gear wheel 23 during the linear backward and forward movement of the drive shafts 15, 15′. The gear wheels 25, 25′ are connected to the gear shafts 15, 15′ by way of suitable fastening means, in this case, as evident from FIG. 4, threaded pins 39 which are introduced into radial drilled holes in a non-toothed portion of the gear wheels 25, 25′, such that a slip-free rotation of the drive shafts 15, 15′ is possible.

The wobble steering ring 14 in the shape of a symmetrical spherical disk has two end faces, as is evident from FIGS. 2 to 6 and in particular FIG. 8. A first end face 28 is aligned in the distal direction and has a first funnel-shaped cutout 28′. The drilled passage holes 30 for threading through the steering wires 12 (shown in FIGS. 3 and 5 to 8) are introduced into a base of the first funnel-shaped cutout 28′, as is a central axial through-opening 14′ of the wobble steering ring 14, which extends from the first end face 28 to a second end face 29 also through the funnel base thereof, with the result that the wobble steering ring 14 can be mounted on the shaft element 21. The second end face 29 of the wobble steering ring 14 comprises a second funnel-shaped cutout 29′. This second funnel-shaped cutout 29′ has a smaller diameter than the first funnel-shaped cutout 28′ and allows tilting on the shaft element 21.

The wobble steering ring 14 is arranged on the shaft element 21 by way of a gimbal-type bearing device that is rotatable with the rod-type shaft 21 about the longitudinal axis B. The bearing device comprises a universal joint plate 10 which is arranged in the central axial through-opening 14′ of the wobble steering ring 14 and on the rod-type shaft 21. The universal joint plate 10, visible in FIGS. 2, 3, and in particular 5, comprises four radial drilled through holes 32 offset by 90° from one another in each case: of these, two coaxial drilled through holes 32 are visible in FIG. 5 and in each case have a bearing pin 34 arranged therein. A respective bearing pin 33 is also arranged in the two further radial drilled through holes, offset by 90° therefrom, of the universal joint plate 10: of these pins, one is visible in FIG. 3 and extends into a drilled through hole 31 in the wobble steering ring 14 which is flush with the corresponding drilled through hole in the universal joint plate 10. Thus, respectively two bearing pins 33, 34 arranged offset by 180° from one another form a first and a second bearing pin pair, wherein the first bearing pin pair, a first bearing pin 33 of which is arranged in the radial drilled through hole 31 of the wobble steering ring 14 in FIG. 3 and both of which bearing pins 33 are visible in FIG. 6, connects the wobble steering ring 14 to the universal joint plate 10. The two further second bearing pins 34, which are arranged offset by 180° from one another, which is to say are coaxial, and which form the second bearing pin pair, connect the universal joint plate 10 to the shaft element 21, wherein the first bearing pin pair of first bearing pins 33 is present offset by 90° from the second bearing pin pair of second bearing pins 34. Hence, the universal joint plate 10 is pivotably arranged on the shaft element 21 by means of the second bearing pins 34 and mounts the wobble steering ring 14 via the bearing pins 33, with the result that said wobble steering ring can be tilted about pivot axes F. F′. Hence, the rotation movement of the wobble steering ring 14 about the longitudinal axis B, actuatable by the steering gear 13, is also transmitted to the shaft element 21 and hence the shaft 2 by way of the gimbal-type bearing device with the universal joint plate 10 and the two bearing pin pairs.

The moving components of the steering gear 13, like ostensibly the wobble steering ring 14 and gear shafts 15, 15′, are embedded in a bearing housing 20 which comprises a base plate 22 arranged below the two gear shafts 15, 15′ and parallel thereto, as shown by FIGS. 2, 3, and 4. In the distal direction, the base plate 22 is adjoined by a first side plate 24 which is present at the end of the contact portions K. K′ of the gear shafts 15, 15′ and which has two drilled passage holes 26 (cf. FIG. 6) for the rotatable mount of the gear shafts 15, 15′. To mount the gear shafts 15, 15′, provision is made by preference of antifriction bearings, for example ball bearings, optionally also plain bearings, which are worked into the drilled passage holes 26. In the proximal direction, the bearing housing 20 comprises a second side plate 24′ with two drilled passage holes 27, through which the drive portion A between the toothing portions 18, 18′ and the gear wheels 25, 25 at the end extends. The second side plate 24′ is attached to the base plate 22 at the end thereof distant from the first side plate 24. Further, the second side plate 24′ comprises a drilled passage hole 26′ for mounting the drive shaft 23′ of the drive gear wheel 23, as is visible in FIG. 5. Mounting to both sides of the shaft element 21 is also depicted there: For the proximal-side mount of the shaft element 21, the second side plate 24′ comprises a further drilled passage hole 27′ with a bearing ring 38. For the distal-side mount of the shaft element 21 in the region of the proximal end 3 of the shaft 2, the housing 20 comprises a third side plate 24″ with a drilled passage hole 27″, in which an antifriction bearing 35 is also arranged.

Further, the second side plate 24′ carries fastening means 37, by means of which the third motor 17″ is fastened to the bearing housing 20. The length of the fastening means 37 corresponds at least to the length of the gear wheel 23, such that the fastening means 37 keep the displacement path of the gear shafts 15, 15′ stable. All three motors 17, 17′, 17″ can be connected to a power source (external or present in the surgical instrument 1) by way of electrical connections 36.

The drawings, the description and the claims contain numerous features in combination. A person skilled in the art will advantageously also consider the features on an individual basis and combine them to form further advantageous combinations. The present disclosure provides a steering gear 13 for a surgical instrument 1 having a shaft 2, at the distal end 5 of which there is a deflection mechanism 9 for manipulating a tool 6, and a surgical instrument 1 having such a steering gear. The steering gear 13 has a shaft element 21 which has a longitudinal axis B and which continues the shaft 2 in the case of an assembled arrangement of the steering gear 13 with the surgical instrument 1. It comprises a wobble steering ring 14 which is operatively couplable to the distal deflection mechanism 9 and which comprises a central axial through-opening 14′ for a bearing device which is secured in the axial longitudinal direction on the shaft element 21 of the steering gear 13 and rotatable about the longitudinal axis B with the shaft element 21. Three motorized drives are present and operatively coupled to the wobble steering ring 14 for transferring the adjustment angles of the three drives to the wobble steering ring 14, wherein the wobble steering ring 14 is spatially alignable in relation to two pivot axes F, F′ running perpendicular to one another and to the longitudinal axis B by way of the adjustment angles of two of the three drives and rotatable about the longitudinal axis B by way of the adjustment angle of the third of the three drives.

1. A steering gear for a surgical instrument having a shaft, at the distal end of which there is a deflection mechanism for manipulating a tool, wherein the steering gear is arrangeable at the proximal end of the shaft, which has a longitudinal axis, and the steering gear comprises:
a shaft element which likewise has a longitudinal axis and which continues the shaft in the case of an assembled arrangement of the steering gear with the surgical instrument; and
a wobble steering ring which is operatively couplable to the distal deflection mechanism for controlling the deflection of same and which comprises a central axial through-opening for a bearing device which is secured in the axial longitudinal direction on the shaft element of the steering gear and rotatable about the longitudinal axis with the shaft element, and
three motorized drives and provides an operative coupling of the three drives to the wobble steering ring for transferring the adjustment angles of the three drives to the wobble steering ring, wherein the wobble steering ring is spatially alignable in relation to two pivot axes running perpendicular to one another and to the longitudinal axis by way of the adjustment angles of two of the three motorized drives and rotatable about the longitudinal axis by way of the adjustment angle of the third of the three motorized drives.

2. The steering gear as set forth in claim 1, wherein the bearing device of the wobble steering ring rotatable with the shaft element about the longitudinal axis is a gimbal-type bearing device which includes a universal joint plate which is arranged in the central axial through-opening and includes four radial drilled through holes offset by 90° from one another in each case, wherein a bearing pin is arranged in each radial drilled through hole wherein two first bearing pins arranged coaxially with respect to one another in the drilled through holes are connected to the wobble steering ring and two second bearing pins arranged coaxially with respect to one another in the drilled through holes offset by 90° are connected to the shaft element.

3. The steering gear as set forth in claim 1, wherein the wobble steering ring has a spherical disk-shaped outer lateral surface as effecting surface, and
the steering gear includes two longitudinally axially displaceable gear shafts, each with a contact roller portion with a force transmitter, wherein the gear shafts each have an axis of rotation which is parallel to the longitudinal axis and spans a center angle of 90° with the longitudinal axis, and
wherein the respective contact roller portion contacts the effecting surface of the wobble steering ring at a contact point, wherein the two pivot axes are each defined by a straight line which runs through the center of the spherical disk-shaped outer lateral surface located on the longitudinal axis and through the respective contact point of the respective contact roller portion with the effecting surface.

4. The steering gear as set forth in claim 3, wherein each gear shaft has a drive portion which includes a toothed rod portion (18, 18′) with teeth all around the circumference, which defines an axial displacement path of the gear shaft (15, 15′) and meshes with a pinion (19, 19′) seated on a drive shaft (17a, 17b) of the respective motor (17, 17′), wherein the drive shafts (17a, 17b) define drive axes (C, C′) which run perpendicular to the longitudinal axis and the axes of rotation of the gear shafts (15, 15′).

5. The steering gear as set forth in claim 3, wherein the two gear shafts are driven to rotate in the same sense about the respective axis of rotation by the third motor; and
wherein each gear shaft includes a gear wheel at a free end of the drive portion, the gear wheel meshing with a common drive gear wheel driven by the third motor by way of a drive shaft parallel to the longitudinal axis and the axes of rotation.

6. The steering gear as set forth in claim 5, wherein an axial length of the drive gear wheel is matched to the axial displacement path of the gear shaft.

7. The steering gear as set forth in claim 3, wherein a force transmission between the contact roller portions of the gear shafts and the effecting portion of the wobble steering ring is provided by friction, wherein at least one of the friction partners of the effecting portion of the wobble steering ring and the respective contact roller portion includes a cladding made of a friction-increasing material or consists of a friction-increasing material, which is preferably an elastomer.

8. The steering gear as set forth in claim 3, wherein the steering gear includes a bearing housing which includes a base plate arranged below the two gear shafts and parallel to the latter, and which
includes a first side plate fastened to the base plate at the end of the contact portions of the gear shafts and including two drilled passage holes for mounting the gear shafts (15, 15′), and
includes a second side plate with two drilled passage holes (27), through which the drive portion located between the toothing portions (18, 18′) and the gear wheels (25, 25′) at the end extends and which is fastened to the base plate at the end of the latter distant from the first side plate.

9. The steering gear as set forth in claim 8, wherein the second side plate carries fastening means (37), by means of which the third motor is fastened to the bearing housing.

10. The steering gear as set forth in claim 1, wherein the wobble steering ring includes:
a first end face which has a first funnel-shaped cutout, wherein drilled passage holes for leading through steering wires are present in a base of the first funnel-shaped cutout and wherein the central axial through-opening of the wobble steering ring extends through the base of the funnel; and
a second end face which includes a second funnel-shaped cutout which has a smaller diameter than the first funnel-shaped cutout, wherein the central axial through-opening further extends through the funnel base of the second funnel-shaped cutout.

11. A surgical instrument comprising:
a shaft;
a manipulation unit arranged at the proximal end of the shaft; and
a tool arranged at the distal end of the shaft with a tool tip able to be deflected by means of a distal deflection mechanism; and
wherein the tool tip able to be deflected is spatially alignable by way of a steering gear as set forth in claim 1.
